(19) United States
(12) Patent Application Publication
(10) Pub. No.: US 2002/0165517 A1
(43) Pub. Date: Nov. 7, 2002

(54) Prefastened diaper/pant for infants with improved fit range

(75) Inventors: Paul Joseph Datta, Appleton, WI (US); Thomas Harold Roessler, Menasha, WI (US); Mark George Everson, Neenah, WI (US); Catherine Marguerite Hancock-Cooke, Neenah, WI (US); Linda Mary Dorschner, Kaukauna, WI (US); Barbara Ann Gossen, Oshkosh, WI (US)

Correspondence Address: Eric T. Krischke, Pauley Petersen Kinne & Erickson, Suite 365, 2800 West Higgins Road, Hoffman Estate, IL, 60195, US

(73) Assignee: Kimberly-Clark Worldwide, Inc.

(21) Appl. No.: 10/026,122

(22) Filed: Dec. 17, 2001

Related U.S. Application Data
(60) Provisional application No. 60/272,548, filed on Mar. 1, 2001.

(51) Int. Cl. A61F 13/15

(52) U.S. Cl. 604/385.22; 604/391; 604/385.28

(57) ABSTRACT

A pant-like, prefastened, disposable absorbent article having elastic and/or extensible components to provide a Percent Fit Range of at least about 33%, more desirably about 80% to about 150%. The disposable absorbent article has prefastened fasteners to allow for easy donning of the article. After initial donning of the article, a waist size adjustment feature allows the waist perimeter dimension to be adjusted to conform the waist opening to the waist of the wearer.

### CROSS REFERENCE TO RELATED APPLICATION

This application claims the benefit of U.S. Provisional Application No. 60/272,548, filed Mar. 01, 2001.

### FIELD OF INVENTION

The present invention relates to disposable absorbent articles that are adapted to contain body exudates. More particularly, the present invention relates to pant-like prefastened disposable absorbent articles having elastic and/or extensible components combinable to provide an increased fit range of the pant-like prefastened, disposable absorbent article.

### BACKGROUND OF THE INVENTION

Absorbent articles such as diapers, training pants or incontinence garments desirably provide a close, comfortable fit about the wearer and contain body exudates. Moreover, absorbent articles, after being soiled, optimally can be removed from the wearer in a convenient and clean manner without undesirably soiling the caregiver, the surrounding area or the clothes of the wearer. In certain circumstances, it is also beneficial for such absorbent articles to be capable of being pulled up or down over the hips of the wearer to allow the wearer or caregiver to easily pull the article on and easily remove the article if it has not been soiled. Given the various shapes and sizes of wearers of absorbent articles, it would also be beneficial for the caregiver or the wearer to be able to adjust the fit of the waist opening of the article.

Conventional diapers are not provided in a prefastened condition and typically include a front waist portion and a back waist portion that are releasably connected about the hips of the wearer by conventional fasteners such as adhesive tape fasteners or hook and loop fasteners. For example, conventional fasteners typically included a pair of fasteners, such as adhesive tape tabs, located on the outermost corners of the diaper in the back waist region of the diaper and a complimentary fastener, such as a taping panel, located on the exterior surface of the outer cover of the diaper in the front waist portion of the diaper. In such a configuration, the diaper is positioned between the legs of the wearer while the wearer is lying down, and the adhesive tape tabs are releasably attached to the taping panel to secure the back waist portion to the front waist portion of the diaper, to secure the diaper about the waist of the wearer. Such conventional training pants are not easily fastened about and removed from the wearer after use without undesirably soiling the caregiver. Further, such conventional diapers are not provided in a pant-like, prefastened configuration and, thus, are not configured to be pulled up or down over the hips of the wearer when the fasteners are attached. Moreover, the fasteners on such conventional diapers generally are difficult to disengage and reattach to further adjust or conform the waist opening of the diaper to the waist of the wearer. Such disengagement and reattachment can also be difficult to accomplish when the wearer is active.

Several attempts have been made to provide absorbent articles that effectively contain body exudates, are capable of being pulled up and/or down over the hips of the wearer and provide for easy cleaning and removal after being soiled. For example, some conventional absorbent articles, such as conventional training pants, include integral side panels that connect the front waist portion to the back waist portion of the absorbent article.

However, many of these absorbent articles have not been completely satisfactory. For example, absorbent articles such as training pants have not always been able to achieve a close conforming fit to the wearer while still being able to expand enough to be pulled up and down over the hips of the wearer. Often such training pants fit the waist of the wearer loosely, which can undesirably result in leaks. As a result, many of these articles have not contained bodily exudates as effectively as conventional diaper-type articles. Moreover, the inspection and removal of soiled absorbent articles that have integral side panels, such as conventional training pants, have not always been completely satisfactory. For example, the side panels are difficult to tear when attempting to remove the article from the waist of the wearer instead of pulling the article down over the hips of the wearer.

Accordingly, despite the attempts to develop improved absorbent articles, there remains a need for absorbent articles that are adjustable to conform to the wearer for effective containment of bodily exudates, are capable of being pulled up and/or down over the hips and buttocks of the wearer, are readily secured about and removed from the wearer in a convenient and clean manner and are easily inspected by the caregiver to determine whether the article is soiled.

### SUMMARY OF THE INVENTION

In response to the difficulties and problems discussed above, new pant-like prefastened, disposable absorbent articles having a waist size adjustment means for an increased fit range have been discovered. Wearers of absorbent articles have a wide range of shapes and sizes. As babies and young children progress through the “step” sizes of absorbent articles, there is an increasing degree of differences in the body shapes of users in the higher step sizes. As the mobility and activity level of a child increases, there are advantages to having access to a pant-like prefastened, disposable absorbent article that can be applied and removed while the child is standing up.

Desirably, the pant-like prefastened, disposable absorbent articles of the invention have a Percent Fit Range that is greater than about 33%, more desirably between about 80% and about 150%. By increasing the fit range of an absorbent article, the number of sizes offered to consumers can be reduced.

The present invention encompasses several approaches to achieving a desired Percent Fit Range greater than about 33%, and more desirably about 80% to about 150%. Delivery of an acceptable fit range is the result of several factors working in unison. Maximizing the use of elastic and/or extensible materials is desirably combined with minimizing the restriction of such materials through design. Desirably, an absorbent article having a Percent Fit Range greater than about 33% can be achieved by “mixing and matching” various components and carefully selecting how the components are assembled together. For example, an absorbent article having elastic ear portions and an elastic waistband can also have one or more alternative components and/or features that improve Percent Fit Range. The alternative components and/or features include, but are not limited to, the following components and features which do not limit, and desirably increase, extensibility at least in the lateral direction of the absorbent article. The lateral direction of an absorbent article having a pant-like or diaper-like configuration is the direction generally parallel to the waistline of the article. The longitudinal direction is the direction generally perpendicular to the waistline, from the front waist region to the back waist region of the article, through the crotch region.

The alternative components and/or features include, but are not limited to, the following components and features:

1. extensible containment flaps that do not restrict extensibility in the waist regions so that a backsheet and a bodyside liner can extend for a wide range of sizes. For example, the containment flaps can be made extensible through use of an elastic or necked nonwoven material (necked stretched in the longitudinal direction of the article for improved lateral extensibility), mechanically sectioning the containment flaps, or tacking down the containment flaps in a limited area so that extensibility is not lost in the waist regions of the absorbent article;

2. a fastener feature having two laterally opposing hook-type fasteners which are each prefastened to a loop component of the fastener feature, wherein the hook-type fasteners are spaced apart from each other by at least about 2.0 inches, but desirably less than about 5.0 inches, in a lateral direction with respect to the loop component in order to permit users of the articles to pull the articles on without first unfastening the fastener feature;

3. a loop component of the fastener feature that is extensible or elastic in the lateral direction of the absorbent article, to permit a front waist region and/or a back waist region to extend laterally beyond a width of a chassis portion of the article, to provide for a larger fit range;

4. a surge layer and/or a tissue layer (that may have distributive or barrier functions) that are necked, pleated or otherwise designed to have extensibility or elasticity in the lateral direction of the absorbent article without tearing;

5. a spacer layer positioned between a backsheet or outer cover and an absorbent layer, the spacer layer being laterally extensible or adhered to the backsheet in a limited area to prevent the spacer layer from restricting extensibility of the backsheet. For example, the spacer layer may be attached to the backsheet over less than about half of a width of the spacer layer;

6. a backsheet and a bodyside liner material that are extensible or elastic in both the longitudinal direction and the lateral direction of the absorbent article;

7. assembling the absorbent article with narrow bonds between the layers using well known bonding techniques such as ultrasonics and adhesives, wherein the bond widths are desirably less than about 50 mm;

8. pleating or bellowing non-extensible materials, such as the chassis, so that the non-extensible materials unfold as the extensible and/or elastic materials elongate as the absorbent article is positioned on the wearer; and

9. application of the waistband, wherein the waistband is applied to the absorbent article at less than stretched to stop elongation.

In accordance with one embodiment of this invention, the waist size adjustment means furnishes a mechanism to reduce the waist perimeter dimension to better maintain the absorbent article about the waist of the wearer, after the absorbent article has been positioned about the hips of the wearer. The waist size adjustment means is intended to maintain the absorbent article in a close conforming fit about the waist of the wearer to reduce the leakage of body exudates. The waist size adjustment means may also be selectively disengaged to facilitate inspection of the absorbent article to determine if it has been soiled.

The waist opening of the prefastened absorbent article desirably can be sufficiently large to allow the prefastened absorbent article to be pulled over the hips of the wearer. However, the circumference of the waist of the wearer is typically less than the circumference of the hips of the wearer. Thus, the waist opening of the prefastened absorbent article may not conform to the waist of the wearer, which may undesirably result in leaks. Hence, the waist size adjustment means is configured to conform the waist regions of the absorbent article to the waist of the wearer, after the prefastened absorbent article is pulled onto the wearer. When the absorbent article must be removed from the wearer, the waist size adjustment means is simply disengaged, if necessary, and the prefastened absorbent article is pulled down over the hips and legs of the wearer.

As a result, the prefastened absorbent articles of the present invention are designed to conform to the body of the wearer to effectively contain bodily exudates while still being capable of being reliably pulled up and/or down over the hips of the wearer to assist in the toilet training of the wearer. Moreover, similar to conventional diapers, the absorbent articles of the present invention can advantageously be applied to and removed from the wearer with relative ease and cleanliness. Removal of the used diaper by opening the fasteners and having the wearer lay on a horizontal surface provides the best opportunity for a clean removal without spills. Removal as a pant by pulling the diaper down over the hips and legs of the wearer can create quite a mess requiring additional cleaning of the wearer and the immediate area.

### DEFINITIONS

As used herein, the term “convertible” refers to an absorbent article that can be pulled on like a pant or applied as a diaper. The article can also be removed as either a pant or a diaper. The user has the option of application/removal that suits the situation.

As used herein, the term “disposable” refers to articles which are intended to be discarded after a limited use and that are not intended to be laundered or otherwise restored for reuse.

As used herein, the term “elastic” or “elastomeric” refers to that property of a material where upon removal of an elongating force, the material is capable of substantially recovering its original size and shape or the material exhibits a significant retractive force.

As used herein, the term “extensible” or “extensible material” refers to that property of a material where upon removal of an elongating force, the material experiences a substantially permanent deformation or the material does not exhibit a significant retractive force.

As used herein, the term “inelastic” refers to materials which are not elastomeric, either because they cannot be sufficiently stretched or because they do not sufficiently retract when stretched and relaxed.

As used herein, the terms “necked” and “neck stretched” are interchangeable terms that refer to a method of elongating an inelastic nonwoven fabric, generally in the longitudinal, or machine direction of the fabric, to reduce its width in a controlled manner to a desired amount. The controlled stretching may take place under cool, room temperature or greater temperatures and is limited to an increase in overall dimension in the direction being stretched up to the elongation required to break the fabric, which in most cases is about 1.2 to 1.4 times. The resulting neck-stretched fabric can be extended in the lateral (cross-machine) direction of the fabric during subsequent use, causing the fabric to return toward its original pre-necked configuration. Neck stretching processes are disclosed, for example, in U.S. Pat. No. 4,443,513 to Meitner et al.; U.S. Pat. No. 4,965,122, U.S. Pat. No. 4,981,747 and U.S. Pat. No. 5,114,781 to Morman; and U.S. Pat. No. 5,244,482 to Hassenboehler Jr. et al.

As used herein, the term “passive bond” refers to a bond which has a relatively low peel strength such that the bond can be easily broken by hand if desired to assist in inspecting or removing an absorbent article from the wearer, without tearing or severely damaging the other portions of the article, and without causing trauma to the wearer or spillage of waste materials from the absorbent article.

As used herein, the term “permanently bonded” refers to the joining, adhering, connecting, attaching, or the like, of two elements of an absorbent article such that the elements tend to be and remain bonded during normal use conditions of the absorbent article.

As used herein, the term “prefastened” refers to a condition wherein the absorbent article has a fastening feature which is engaged or fastened prior to use by the wearer. For example, the fastening feature of the absorbent article may be engaged or fastened during the manufacturing process.

As used herein, the term “refastenable” refers to the property of two elements being capable of releasable attachment, separation, and subsequent releasable reattachment without substantial permanent deformation or other damage to either element.

As used herein, the term “releasably attached,” “releasably engaged” and variations thereof refer to two elements being connected or connectable such that the elements tend to remain connected absent a separation force applied to one or both of the elements, and the elements being capable of separation without substantial permanent deformation or other damage. The required separation force is typically beyond that encountered while wearing the absorbent article.

As used herein, the term “stretchable” refers to a material that may either be extensible or elastic. That is, the material may be extended, deformed or the like, without breaking, and may or may not significantly retract after removal of an extending force.

As used herein, the term “tension” refers to a uniaxial force tending to cause the extension of a body or a balancing force within that body resisting the extension.

### BRIEF DESCRIPTION OF THE DRAWINGS

The present invention will be more fully understood and further advantages will become apparent when reference is made to the following detailed description of the invention and the accompanying drawings wherein like numerals represent like elements. The drawings are merely representative and are not intended to limit the scope of the appended claims.

FIG. 1 representatively shows a perspective view of a pant-like prefastened, disposable absorbent article, according to one embodiment of this invention;

FIG. 2 representatively shows a front plan view of the pant-like prefastened, disposable absorbent article of FIG. 1, according to one embodiment of this invention;

FIG. 3 representatively shows a plan view of the pant-like prefastened, disposable absorbent article of FIG. 1 in an unfastened, stretched and laid flat condition with a surface of the article which contacts the wearer's skin facing the viewer, according to one embodiment of this invention;

FIG. 4 representatively shows a perspective view of a pant-like prefastened, disposable absorbent article, according to one embodiment of this invention;

FIG. 5 representatively shows a front plan view of the pant-like prefastened, disposable absorbent article of FIG. 4, according to one embodiment of this invention;

FIG. 6 representatively shows a front plan view of the pant-like prefastened, disposable absorbent article of FIG. 4 with a waist size adjustment means in an unengaged position, and with portions of the article partially cut away to show the underlying features, according to one embodiment of this invention;

FIG. 7 representatively shows a plan view of the pant-like prefastened, disposable absorbent article of FIG. 4 in an unfastened, stretched and laid flat condition with a surface of the article which contacts the wearer's skin facing the viewer, according to one embodiment of this invention;

FIG. 8 representatively shows a front plan view of a pant-like prefastened, disposable absorbent article having a waist size adjustment means in an unengaged position, and with portions of the article partially cut away to show the underlying features, according to one embodiment of this invention; and

FIG. 9 representatively shows a front plan view of a pant-like prefastened, disposable absorbent article having a passive bond forming a “zig-zag” bond pattern, according to one embodiment of this invention.

### DETAILED DESCRIPTION OF THE INVENTION

The present invention is directed to a pant-like prefastened, disposable absorbent article adaptable to be worn adjacent to the body of a wearer to absorb and contain various exudates discharged from the body. The absorbent article is initially fastened, or prefastened, so the absorbent article can be pulled over the wearer's hips and buttocks and positioned properly to effectively absorb and/or contain the body exudates. The prefastened absorbent article is also refastenable such that the absorbent article can be adjusted or fitted to the wearer's waist size and easily inspected to determine if the absorbent article has been soiled. For example, the pant-like prefastened, disposable absorbent article may include a waist size adjustment means which allows the wearer or the caregiver to adjust the fit of the article once it has been positioned on the hips of the wearer, without disengaging the prefastened side seams. As such, the pant-like prefastened, disposable absorbent article of the present invention can function in a similar manner to conventional training pants when left in the prefastened configuration, or can be unfastened prior to or during use to function in a refastenable manner similar to a conventional diaper.

The pant-like prefastened, disposable absorbent article of the present invention has an improved fit range performance when an elastic or extensible bodyside liner and outer cover are matched with other materials that do not inhibit the extension and/or retraction properties of the elastic and/or extensible components. The fit range performance of the absorbent article typically is measured in Percent Fit Range. Desirably, the Percent Fit Range of the absorbent article is at least about 33%, more desirably about 80% to about 150%. The Percent Fit Range (“PFR”) of the absorbent article is measured using the following equation:

$$PFR = \frac{{WP}_{e} - {WP}_{r}}{{WP}_{r}} \times 100 ; \qquad \text{Eq.} ( 1 )$$

wherein WPₑ is an extended waist performance measurement of the absorbent article and WPᵣ is a relaxed waist performance measurement of the absorbent article. When measuring the Percent Fit Range of the absorbent article, the fasteners, discussed below, can be positioned about 2.5 inches apart from each other along one of the front waist region and the back waist region of the article. The extended waist performance measurement and the relaxed waist performance measurement of the absorbent article are dependent upon many factors, including the age and size of the wearer. The extended waist performance measurement is measured when the absorbent article is extended in the lateral direction by a force of about 1400 grams. This force is applied using conventional testing procedures. This applied force is comparable to a maximum comfortable tension force of the absorbent article against the wearer's waist during use. The relaxed waist performance measurement is measured when the absorbent article is laterally extended by a force of about 80 grams to about 100 grams. This applied force is comparable to a minimum tension force required to maintain the absorbent article in proper wearing position on the wearer. In comparison, a typical tension force applied to the absorbent article during use is about 400 grams.

To provide a desired Percent Fit Range of at least about 33%, the absorbent article must have components which are elastic or extensible in at least one desired direction, for example in a lateral or cross-machine direction. Cost-effective elastic materials, as well as additional extensible materials which do not limit or restrict the elastic materials, determine the Percent Fit Range of the absorbent article.

The pant-like prefastened, disposable absorbent article of the present invention will be described in terms of a pant-like prefastened, disposable diaper that is adapted to be worn by infants about the lower torso. In particular, the pant-like prefastened, disposable absorbent article will be described in terms of a pant-like prefastened, disposable diaper 20 having a waist size adjustment means. It is apparent that the articles and methods of the present invention are equally adaptable for other types of absorbent articles such as adult incontinent products, training pants, feminine hygiene products, other personal care or health care garments, and the like.

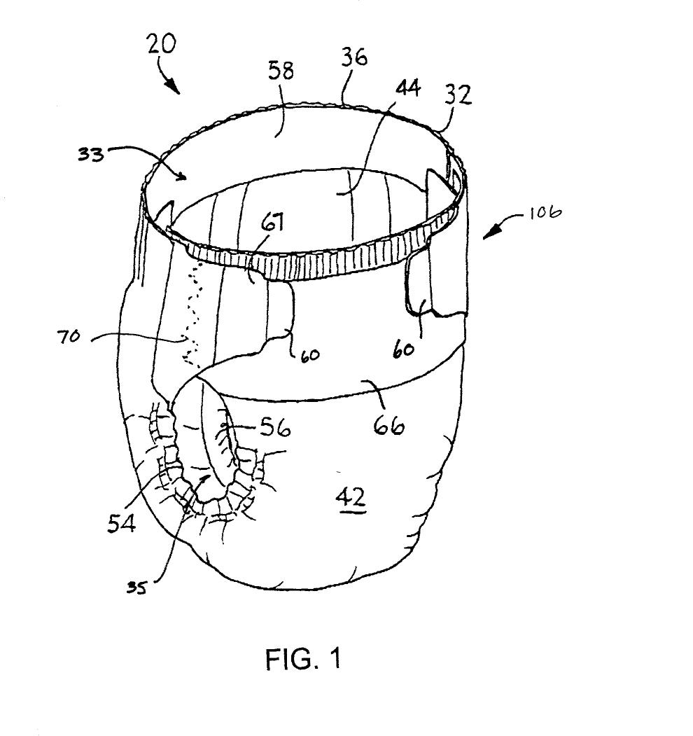

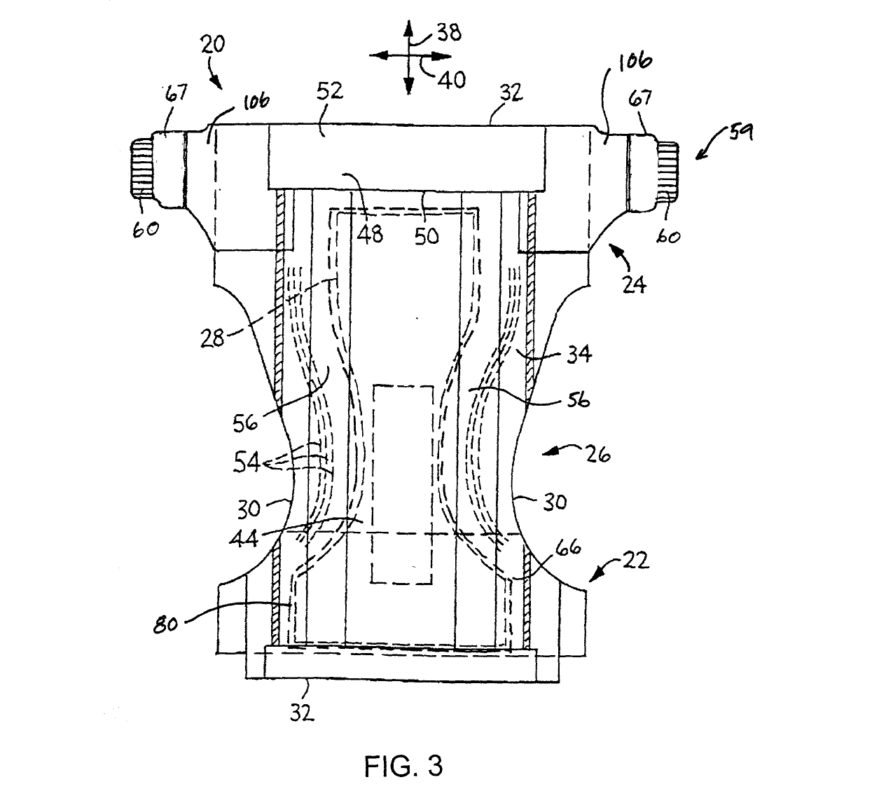

Referring to FIGS. 1-9, a pant-like prefastened, disposable diaper 20 of the present invention defines a front waist region 22, a back waist region 24, and a crotch region 26 that extends between and connects the front waist region 22 and the back waist region 24. For reference, arrows 38 and 40 depicting the orientation of the longitudinal axis and the lateral axis, respectively, of the diaper 20 are illustrated in FIGS. 3 and 6-8.

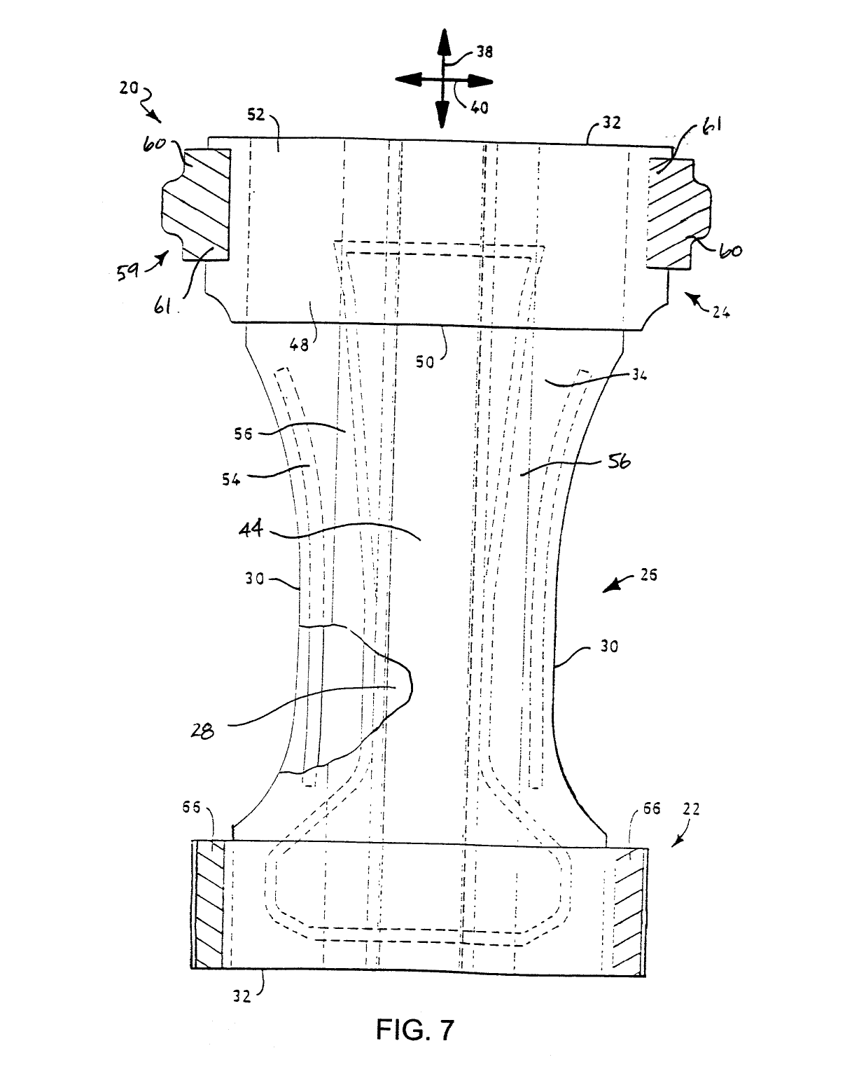

As shown in FIGS. 3 and 7, the front waist region 22 includes the portion of the diaper 20 that, when worn, is positioned on the front of the wearer while the back waist region 24 includes the portion of the diaper 20 that, when worn, is positioned on the back of the wearer. The crotch region 26 of the diaper 20 includes the portion of the diaper 20 that, when worn, is positioned between the legs of the wearer and covers the lower torso of the wearer.

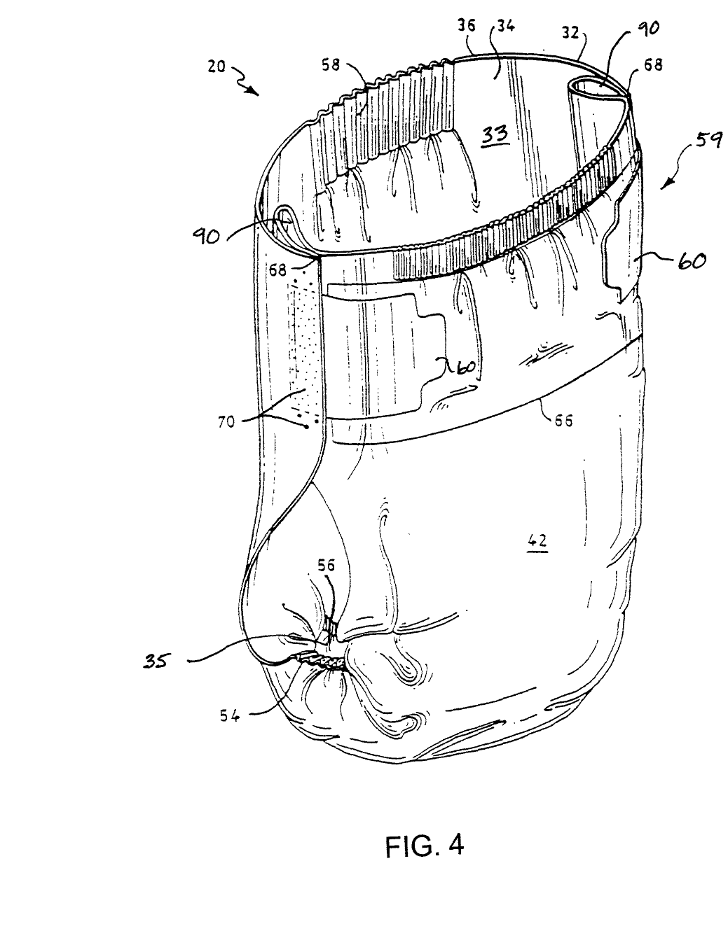

The diaper 20 defines a pair of laterally opposing side edges 30, a pair of longitudinally opposing waist edges 32, an interior surface 34 that is configured to contact the wearer, and an exterior surface 36 opposite the interior surface 34 that is configured to contact the wearer's clothing during use. Referring to FIGS. 1 and 4, the diaper 20 also includes an outer cover 42 and a bodyside liner 44 that is connected to the outer cover 42 in a superposed relation to define a chassis having a waist opening 33 and two leg openings 35. An absorbent core 28 is located between the outer cover 42 and the bodyside liner 44. The laterally opposing side edges 30 are generally defined by the side edges of the outer cover 42 that further define leg openings which desirably are curvilinear. The waist edges 32 are generally defined by the waist edges of the outer cover 42 and form the waist opening 33 that is configured to encircle the waist of the wearer during use. The absorbent core 28 is configured to contain and/or absorb any body exudates discharged from the wearer.

The diaper 20 may be of various suitable shapes. For example, in the unfastened configuration as shown in FIGS. 3 and 7, the diaper 20 may have an overall rectangular shape, T-shape or an approximately hourglass shape. In the shown embodiment, the diaper 20 has a generally I-shape in an unfastened configuration. Examples of suitable diaper configurations and suitable diaper components are described in U.S. Pat. No. 4,798,603, issued Jan. 17, 1989 to Meyer et al.; U.S. Pat. No. 5,176,668, issued Jan. 5, 1993 to Bemardin; U.S. Pat. No. 5,176,672, issued Jan. 5, 1993 to Bruemmer et al.; U.S. Pat. No. 5,192,606, issued Mar. 9, 1993 to Proxmire et al.; and U.S. Pat. No. 5,509,915, issued Apr. 23, 1996 to Hanson et al., the disclosures of which are herein incorporated by reference. The various components and configurations of the diaper 20 according to embodiments of this invention provide a desired fit range, as well as softness, body conformity, reduced red-marking of the wearer's skin, reduced skin hydration, improved containment of body exudates and improved aesthetics.

The various components of the diaper 20 are integrally assembled together employing various types of suitable attachment means, such as adhesive, sonic and thermal bonds and combinations thereof. In the shown embodiment, for example, the outer cover 42 and bodyside liner 44 are assembled to each other and to the absorbent core 28 with an adhesive, such as a hot melt, pressure-sensitive adhesive. The adhesive may be applied as a uniform continuous layer of adhesive, a patterned layer of adhesive, a sprayed pattern of adhesive, or an array of separate lines, swirls or dots of adhesive. Similarly, the other components of the diaper 20 may be assembled into the diaper 20 by employing the above-identified attachment mechanisms.

In accordance with one embodiment of this invention, as shown in FIGS. 1-9, the outer cover 42 is extensible, for example as described in U.S. patent application Ser. No. 09/563,417 filed on May 3, 2000 by Roessler et al., the disclosure of which is incorporated herein by reference to the extent it assists the present disclosure. Other suitable extensible outer covers 42 are described in U.S. patent application No. 09/460,490 filed on Dec. 14, 1999 by Morman et al. and U.S. patent application Ser. No. 09/751,414 filed on Dec. 28, 2000 by Morman et al., the disclosures of which are incorporated herein by reference to the extent each assists the present disclosure.

The outer cover 42 of the diaper 20, for example as shown in FIGS. 1 and 4, may suitably be composed of a material which is either water vapor permeable or water vapor impermeable. It is generally desired that the outer cover 42 be formed from a material that is substantially impermeable to liquids and permeable (breathable) to water vapor. The outer cover 42 can be manufactured from a thin plastic film or other suitable flexible liquid-impermeable material. For example, the outer cover 42 may be formed from a polyethylene film having a thickness of about 0.013 millimeter (0.5 mil) to about 0.051 millimeter (2.0 mils). If a more clothlike feeling is desired, the outer cover 42 may be formed from a polyolefin film having a nonwoven web, such as a spunbond web of polyolefin fibers, laminated to an exterior surface thereof. For example, a stretch-thinned polypropylene film having a thickness of about 0.015 millimeter (0.6 mil) may have thermally laminated thereto a spunbond web of polypropylene fibers. The polypropylene fibers may have a thickness of about 1.8 to 2.5 denier per filament, which nonwoven web has a basis weight of about 17 grams per square meter (0.5 ounce per square yard). In certain embodiments, the fibers may have a thickness of up to about 3.2 denier per filament. The outer cover 42 may otherwise include bicomponent fibers such as polyethylene/polypropylene bicomponent fibers. Methods of forming such clothlike outer covers are known to those skilled in the art.

Further, the outer cover 42 may be formed of a woven or nonwoven fibrous web layer which has been totally or partially constructed or treated to impart a desired level of liquid impermeability to selected regions that are adjacent or proximate the absorbent core 28. Still further, the outer cover 42 may optionally be composed of a micro-porous “breathable” material which permits vapors to escape from the absorbent core 28 while still preventing liquid exudates from passing through the outer cover 42. For example, the outer cover 42 may include a vapor permeable non-woven facing layer laminated to a micro-porous film. Suitable “breathable” outer cover materials are described in U.S. Pat. No. 5,695,868, issued to McCormack et al.; and U.S. Pat. No. 5,843,056, issued Dec. 1, 1998 to Good et al., the disclosures of which are hereby incorporated by reference. Still further, the outer cover 42 may also be an elastomeric material such as a stretch-thermal laminate (STL), a neck-bonded laminate (NBL), or a stretch-bonded laminate (SBL) material. Methods of making such materials are well known to those skilled in the art and are described in U.S. Pat. No. 4,663,220, issued May 5, 1987 to Wisneski et al.; U.S. Pat. No. 5,226,992, issued Jul. 13, 1993 to Mormon; and European Patent Application No. EP 0 217 032 published on Apr. 8, 1987 in the names of Taylor et al., the disclosures of which are hereby incorporated by reference. The outer cover 42 can also be embossed or otherwise provided with a matte finish to provide a more aesthetically pleasing appearance.

As shown in FIGS. 3 and 7, the bodyside liner 44 suitably presents a bodyfacing surface that is compliant, soft feeling, and nonirritating to the wearer's skin. In accordance with one embodiment of this invention, the bodyside liner 44 is desirably made of an elastic and/or an extensible material. For example, the bodyside liner 44 may be made from extensible materials as are described in U.S. patent application Ser. No. 09/563,417 filed on May 3, 2000 by Roessler et al. Desirably, these materials are elastic or extensible in at least a cross-machine direction, parallel to the lateral axis depicted by arrow 40 in FIG. 7.

Further, the bodyside liner 44 may be less hydrophilic than the absorbent core 28, to present a relatively dry surface to the wearer, and may be sufficiently porous to be liquid permeable, permitting liquid to readily penetrate through its thickness. Suitable web materials for manufacturing the bodyside liner 44 include, but are not limited to, porous foams, reticulated foams, apertured plastic films, natural fibers (for example, wood or cotton fibers), synthetic fibers (for example, polyester or polypropylene fibers), and/or a combination of natural and synthetic fibers. The bodyside liner 44 is suitably employed to help isolate the wearer's skin from liquids held in the absorbent core 28.

Various woven and nonwoven fabrics can be used for the bodyside liner 44. For example, the bodyside liner 44 may be composed of a meltblown or spunbond web of polyolefin fibers. The bodyside liner 44 may also be a bonded-carded web composed of natural and/or synthetic fibers. The bodyside liner 44 may be composed of a substantially hydrophobic material, and the hydrophobic material may optionally be treated with a surfactant or otherwise processed to impart a desired level of wettability and hydrophilicity. In one embodiment of the invention, the bodyside liner 44 is made from a nonwoven, spunbond, polypropylene fabric composed of an average 2.37 denier fibers formed into a web having a basis weight of about 17 grams per square meter and a density of about 0.0652 grams per cubic centimeter. The fabric may be surface treated with about 0.3 weight percent of a surfactant commercially available from Uniqema Inc., a division of ICI of New Castle, Del., U.S.A., under the trade designation AHCOVEL Base N-62. The surfactant can be applied by. The surfactant may be applied by any conventional means, such as spraying, printing, brush coating or the like. The surfactant may be applied to the entire bodyside liner 44 or may be selectively applied to particular sections of the bodyside liner 44, such as the medial section along the longitudinal centerline of the diaper, to provide greater wettability of such sections. The bodyside liner 44 may further include a lotion or treatment applied thereto that is configured to be transferred to the wearer's skin.

The absorbent core 28 of the diaper 20 may suitably be composed of a matrix of hydrophilic fibers, such as a web of cellulosic fluff, mixed with particles of a high-absorbency material commonly known as superabsorbent material. In one embodiment of this invention, the absorbent core 28 includes a matrix of cellulosic fluff such as wood pulp fluff and superabsorbent hydrogel-forming particles. The wood pulp fluff may be exchanged with synthetic, polymeric, meltblown fibers or with a combination of meltblown fibers and natural fibers. The superabsorbent particles may be substantially homogeneously mixed with the hydrophilic fibers or may be nonuniformly mixed. The fluff and superabsorbent particles may also be selectively placed into desired zones of the absorbent core 28 to better contain and absorb body exudates. The concentration of the superabsorbent particles may also vary through the thickness of the absorbent core 28. Alternatively, the absorbent core 28 may comprise a laminate of fibrous webs and superabsorbent material or other suitable means of maintaining a superabsorbent material in a localized area.

The absorbent core 28 may have any suitable shape. For example, the absorbent core 28 may be rectangular, I-shaped, or T-shaped. Desirably, the absorbent core 28 is narrow in the crotch region 26 of the diaper 20. The absorbent core 28 desirably has a width in the crotch region 26 of about 2.5 centimeters to about 12.7 centimeters (1.0 inch to about 5.0 inches), more desirably not greater than about 7.6 centimeters (3.0 inches) and even more desirably not greater than about 5.1 centimeters (2.0 inches). The narrow width of the absorbent core 28 in the crotch region 26 allows the absorbent core 28 to better fit between the legs of the wearer. It is apparent that the dimensions and the absorbent capacity of the absorbent core 28 should properly correspond to the size of the intended wearer and the liquid loading imparted by the intended use of the diaper 20.

Suitable high-absorbency materials for the absorbent core 28 include, but are not limited to, natural, synthetic, and modified natural polymers and materials. The high-absorbency materials can be inorganic materials, such as silica gels, or organic compounds, such as crosslinked polymers. The term “crosslinked” refers to any means for effectively rendering normally water-soluble materials substantially water insoluble but swellable. Such means can include, for example, physical entanglement, crystalline domains, covalent bonds, ionic complexes and associations, hydrophilic associations such as hydrogen bonding, and hydrophobic associations or Van der Waals forces.

Examples of suitable synthetic, polymeric, high-absorbency materials include, but are not limited to, the alkali metal and ammonium salts of poly(acrylic acid) and poly(methacrylic acid), poly(acrylamides), poly(vinyl ethers), maleic anhydride copolymers with vinyl ethers and alpha-olefins, poly(vinyl pyrolidone), poly(vinyl morpholinone), poly(vinyl alcohol), and mixtures and copolymers thereof. Further polymers suitable for use in the absorbent core 28 include, but are not limited to, natural and modified natural polymers, such as hydrolyzed acrylonitrile-grafted starch, acrylic acid grafted starch, methyl cellulose, carboxymethyl cellulose, hydroxypropyl cellulose, and the natural gums, such as alginates, xanthan gum, locust bean gum, and similar compounds. Mixtures of natural and wholly or partially synthetic absorbent polymers can also be useful in the present invention. Such high-absorbency materials are well known to those skilled in the art and are widely commercially available. Examples of superabsorbent polymers suitable for use in the present invention are SANWET IM 3900 polymer available from Hoechst Celanese located in Portsmouth, Va. and DOW DRYTECH 2035LD polymer available from Dow Chemical Co. located in Midland, Mich.

The high absorbency material may be in any of a wide variety of geometric forms. Generally, it is desired that the high absorbency material be in the form of discrete particles. However, the high absorbency material may also be in the form of fibers, flakes, rods, spheres, needles, or the like. Generally, the high absorbency material is present in the absorbent core 28 in an amount of about 5 weight percent to about 90 weight percent, based on a total weight of the absorbent core 28.

In accordance with one embodiment of this invention, a substantially hydrophilic tissue wrapsheet 80, for example as shown in FIG. 1, may be employed to help maintain the integrity of the airlaid fibrous structure of the absorbent core 28. The tissue wrapsheet 80 is typically placed about the absorbent core 28 over at least one of the two major facing surfaces thereof and composed of an absorbent cellulosic material, such as creped wadding or a high wet-strength tissue. In accordance with one embodiment of the invention, the tissue wrapsheet 80 can be configured to provide a wicking layer that helps to rapidly distribute liquid over the mass of absorbent fibers comprising the absorbent core 28. The tissue wrapsheet 80 on one side of the absorbent fibrous mass may be bonded to the tissue wrapsheet 80 located on the opposite side of the fibrous mass to effectively entrap the absorbent core 28. In accordance with one embodiment of this invention, the tissue wrapsheet 80 may be necked, pleated and/or have any suitable design known to those having ordinary skill in the art to extend in at least one direction, for example the lateral or cross-machine direction, without tearing or ripping.

In accordance with one embodiment of this invention as shown in FIGS. 3 and 7, the diaper 20 includes a pair of containment flaps 56 that are configured to provide a barrier to the lateral flow of body exudates. The containment flaps 56 may be located along the laterally opposing side edges 30 of the diaper 20 adjacent the side edges of the absorbent core 28. Each containment flap 56 typically defines an unattached edge that is configured to maintain an upright, perpendicular configuration in at least the crotch region 26 of the diaper 20 to form a seal against the wearer's body. Each containment flap 56 extends longitudinally along at least a portion of a length of the absorbent core 28. Desirably, each containment flap 56 extends along substantially the entire length of the absorbent core 28 to better contain the body exudates. In accordance with one embodiment of this invention wherein each containment flap 56 extends along a portion of the length of the absorbent core 28, the containment flaps 56 can be selectively positioned along the side edges 30 of the diaper 20 in the crotch region 26. Such containment flaps 56 are generally well known to those skilled in the art. For example, suitable constructions and arrangements for containment flaps 56 are described in U.S. Pat. No. 4,704,96, issued Nov. 3, 1987 to K. Enloe, the disclosure of which is hereby incorporated by reference.

In accordance with one embodiment of this invention, each containment flap 56 may be extensible, necked, mechanically sectioned and/or tacked down in a limited area to allow the bodyside liner 44 and the outer cover 42 to extend so that the waist opening 33 of the diaper 20 is adjustable. For example, in one embodiment of this invention, the containment flaps 56 may be tacked down at least about 50 mm from a waistband region of the diaper 20. Alternatively or in addition, the containment flap 56 may be tacked down less than about 35 mm in a CD or lateral direction. Thus, the fit range of the diaper 20 is not negatively affected by the use of the containment flaps 56 in the diaper 20.

In accordance with one embodiment of this invention, at least a portion of the waist edges 32 and the side edges 30 are elastic or extensible to improve the fit range of the diaper 20 and support the absorbent core 28 to prevent leakage of body exudates. For example, referring to FIGS. 1-9, the diaper 20 may include a pair of leg elastic members 54 that are connected to the laterally opposing side edges 30 of the diaper 20 in the crotch region 26. The diaper 20 may also include a pair of waist elastic members 58 that are connected to the longitudinally opposing waist edges 32 of the diaper 20. The leg elastic members 54 and the waist elastic members 58 are generally adapted to fit about the legs and waist of a wearer, respectively, to maintain a positive, contacting relationship with the wearer to effectively reduce or eliminate the leakage of body exudates from the diaper 20.

Materials suitable for use as the leg elastic members 54 and the waist elastic members 58 are well known to those skilled in the art. For example, sheets, strands or ribbons of a polymeric, elastomeric material may be adhered to the outer cover 42 in a stretched position, or attached to the outer cover 42 while the outer cover 42 is pleated, such that elastic constrictive forces are imparted to the outer cover 42. The leg elastic members 54 may also include such materials as polyurethane, synthetic rubber and natural rubber.

In accordance with one embodiment of this invention as shown in FIGS. 3 and 7, the diaper 20 may include a fit panel 48 superimposed adjacent to the waist edge 32 in at least one of the front waist region 22 and the back waist region 24, to provide a more comfortable, contouring fit about the wearer. Desirably, the fit panel 48 is elastic, elastomeric or extensible in at least one direction, such as the cross-machine or lateral direction. For example, as shown in FIGS. 3 and 7, the diaper 20 includes an elastomeric fit panel 48 on the interior surface 34 of the diaper 20 that is configured to elongate in the lateral direction to provide an improved fit range and enhanced appearance of the diaper 20. Desirably, the elastic or extensible fit panel 48 allows the waist opening 33 to be extended or adjusted, thereby increasing the waist perimeter dimension to assist in applying the diaper 20 onto the wearer. The fit panel 48 is also configured with respect to the diaper 20 such that the absorbent core 28 has the ability to move and receive body exudates without adversely affecting the positioning of the fit panel 48 and the diaper 20 about the waist of the wearer. Thus, movements of the wearer may cause the absorbent core 28 to move but do not adversely affect the overall positioning and fit of the diaper 20 on the wearer. Such improved fit can result in reduced leakage from the diaper 20, increased comfort and a more aesthetically pleasing appearance.

As shown in FIG. 7, in accordance with one embodiment of this invention, the fit panel 48 is located on the interior surface 34 and extends longitudinally beyond the side edges of the absorbent core 28 so that the fit panel 48 is generally coterminous with the waist edge 32 in the respective front waist region 22 and/or back waist region 24. When located on the interior surface 34 of the diaper 20, the fit panel 48 may define a free edge 50 that extends longitudinally inward towards the crotch region 26 of the diaper 20. In accordance with one embodiment of this invention, the free edge 50 is configured to remain at least partially unattached to the bodyside liner 44 to allow the absorbent core 28 to move, for example expand, to receive and contain body exudates. The unattached free edge 50 allows a pocket to form between the fit panel 48 and the bodyside liner 44 to contain body exudates. Further, the free edge 50, as well as the waist edge 52, of the fit panel 48 may be linear or curvilinear to better fit the wearer. Desirably, if the free edge 50 is curvilinear, the waist edge 52 is also curvilinear to improve the manufacturing process and reduce manufacturing waste.

Suitable materials for producing the fit panel 48 include, but are not limited to, stretch-bonded-laminate (SBL) materials, neck-bonded-laminate (NBL) materials, elastomeric films, elastomeric foam materials, and/or the like. For example, suitable meltblown elastomeric fibrous webs are described in U.S. Pat. No. 4,663,220, issued May 5, 1987 to T. Wisneski et al., the disclosure of which has previously been incorporated by reference. Examples of suitable composite fabrics comprising at least one layer of a nonwoven material secured to a fibrous elastic layer are described in European Patent Application No. EP 090 010 published on Apr. 8, 1987 with the inventors listed as J. Taylor et al., the disclosure of which has previously been incorporated by reference. Examples of suitable NBL materials are described in U.S. Pat. No. 5,226,992, issued Jul. 13, 1993 to Morman, the disclosure of which has previously been incorporated by reference.

In accordance with one embodiment of this invention, the fit panel 48 may be attached to the diaper 20 in any suitable manner that provides the desired elastic or extensible properties. For example, the fit panel 48 may be attached to the diaper 20 using adhesive, ultrasonic, and/or thermal bonding techniques, and the like.

As shown in FIGS. 1-9, in accordance with one embodiment of this invention, the diaper 20 includes a fastening system 59 having at least one, and desirably two laterally opposing refastenable, prefastened fasteners 60. The fasteners 60 are permanently bonded, adhered or otherwise attached directly or indirectly to the diaper 20 at or laterally inward from its side edges 30, in one of the front waist region 22 or the back waist region 24. The fasteners 60 may be permanently bonded or attached in the vicinity of the side edges 30 of the diaper 20 by any means known to those skilled in the art such as adhesive bonds, sonic bonds or thermal bonds. Desirably, the fasteners 60 are permanently bonded or attached to the back waist region 24 (FIG. 1), having the extensible fit panel 48, using bonds having a width of less than about 0.5 inch. Alternatively, the fasteners 60 may be permanently bonded or attached directly to the extensible fit panel 48 (FIG. 7). Attaching the fasteners 60 onto the front waist region 22 or the back waist region 24 that the extensible fit panel 48 is attached to, or directly to the extensible fit panel 48, provides added fit, flexibility, and an optimum seal in the corresponding front waist region 22 or the back waist region 24 once the diaper 20 is positioned on the wearer.

As shown in FIGS. 1-3, at least one fastener 60 may be permanently bonded or attached in the vicinity of the side edges 30 of the diaper 20 to a side panel or ear 106. In accordance with one embodiment of this invention, at least a portion of the ear 106 may be elastic or extensible in at least one direction, for example the lateral direction. For example, as shown in FIGS. 1-3, the fastener 60 is permanently bonded to an elastic or extensible portion 67 of the back side panel or ear 106, which is connected at the back waist region 24 to the chassis of the diaper 20. The elastic or extensible portion 67 allows ear 106 to be extensible in at least one direction, for example the lateral direction to provide an improved fit range of the diaper 20.

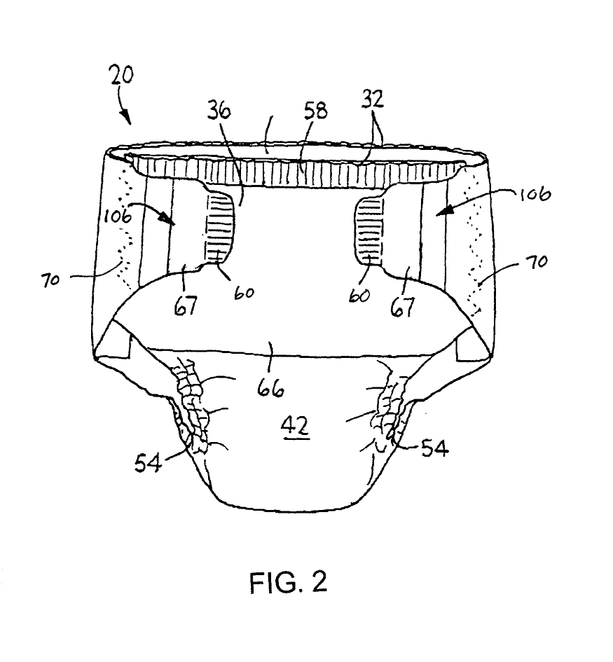

In accordance with one embodiment of this invention, the fastener 60 is refastenably engageable with an attachment panel 66, as shown in FIGS. 1 and 2, whereby the waist perimeter dimension can be adjusted to conform to the waist of the wearer, as discussed below. Desirably, the attachment panel 66 extends laterally across the outer surface 36 of the front waist region 22 and may extend outwardly from the side edges 30 of the chassis, as shown in FIG. 3.

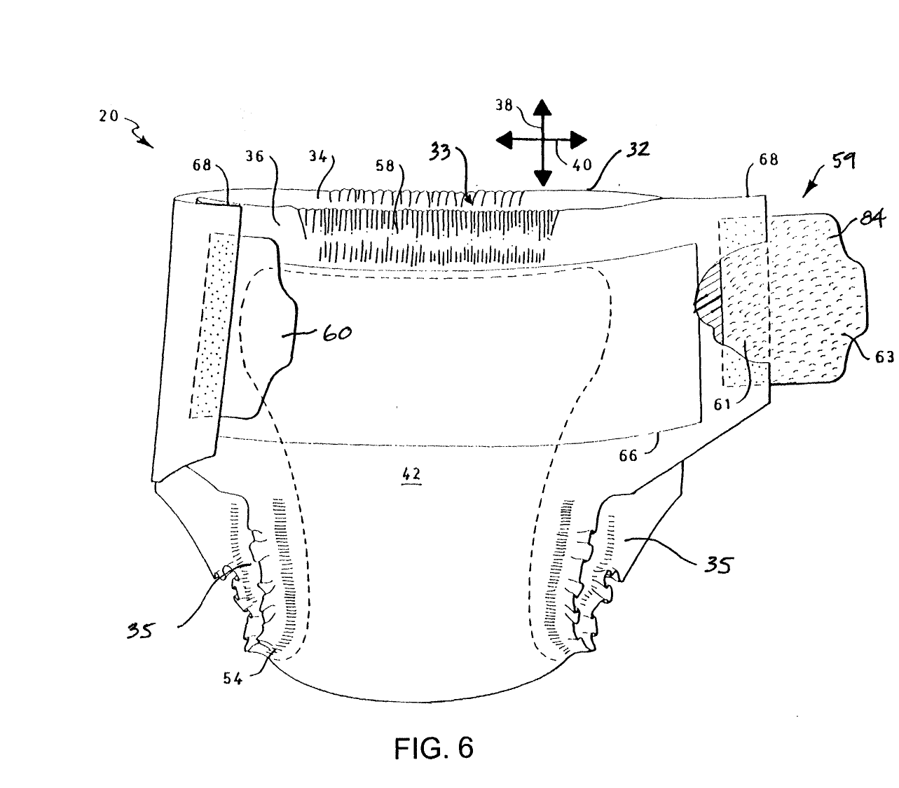

As shown in FIG. 6, in accordance with one embodiment of this invention, at least one of the fasteners 60 may include a unitary, continuous fastening segment having a first engageable portion 61 that provides a prefastened, refastenable side seam 68 and a second engageable portion 63 that provides a waist size adjustment means. The second engaging portion 63 is refastenably engageable with the attachment panel 66 and/or the exterior surface 36 of the diaper, independently of the refastenable side seam 68, whereby the waist perimeter dimension can be adjusted to conform to the waist of the wearer, as discussed below. When the first engageable portion 61 of each fastener 60 is refastenably engaged with the corresponding front waist region 22 and/or back waist region 24, providing the prefastened refastenable side seams 68, the laterally opposing side edges 30 of the diaper 20 each defines a leg opening 35 which is configured to encircle a leg of the wearer. Further, the waist edges 32 define the waist opening 33. The second engageable portion 63 of each fastener 60 is refastenably engageable directly with the outer surface 36 or alternatively with an attachment panel 66 which extends laterally across the outer surface 36 of the front waist region 22, as shown in FIGS. 4-6. The use of the fasteners 60 that provide both the side seam 68 and a waist size adjustment means with one continuous fastening segment advantageously simplifies the manufacturing process and reduces raw material requirements, resulting in reduced manufacturing costs. In certain embodiments of this invention, as shown in FIG. 4, a bellow 90 is formed when the front side panel and/or the attachment panel 66 is folded over to fasten or attach the fastener 60 to the attachment panel 66. The bellows 90 provide added fit range by allowing non-extensible materials to unfold as the elastic and/or extensible materials extend.

In accordance with one embodiment of this invention as shown in FIGS. 4-6, the extensible or elastic attachment panel 66 may include a single piece of material, such as a loop component material, that extends substantially across the respective waist region of the diaper 20. In this configuration, the attachment panel 66 located on the outer cover 42 may further extend beyond the side edges 30 of the diaper 20 and include a folded over portion to which the first engageable portion 61 of continuous fastener 60 is refastenably engaged to provide the prefastened refastenable side seams 68.

Desirably, the second engageable portion 63 is made of a suitable releaseably engageable fastener, such as an adhesive tape tab fastener, hook fastener, loop fastener, mushroom fastener, snap, pin, belt and the like, and combinations thereof. For example, as shown in FIG. 6, the second engageable portion 63 may include a plurality of hook type fasteners and the attachment panel 66 and/or the outer cover 42 may be configured to function as a complimentary loop type fastener.

As shown in FIG. 7, in accordance with one embodiment of this invention, the diaper 20 includes two separate attachment panels 66 located along the opposing side edges 30 on the interior surface 34 in the front waist region 22. The first engageable portion 61 of each fastener 60, which are permanently bonded or attached to the diaper 20 in the opposing waist region, for example the back waist region 24, are releasably engaged to the attachment panels 66 to provide the prefastened, refastenable side seams 68. Alternatively, if the fasteners 60 are located on the front waist region 22, the attachment panels 66 may be located along the interior surface 34 of the diaper 20 in the back waist region 24. The attachment panels 66 may be otherwise located on the exterior surface 36 of the diaper 20 at the front waist region 22 and/or the back waist region 24, depending upon the location of the fasteners 60. In such configurations, the prefastened, refastenable side seams 68 may be formed as described above, provided that the interior surface 34 or the exterior surface 36 of the diaper 20 to which the attachment panels 66 are attached, are configured to be exposed to the first engageable portions 61 of the fastener 60.

Desirably, the side seams 68 are configured such that the exterior surface 36 of the front waist region 22 is refastenably attached to the interior surface 34 of the back waist region 24. As such, multiple benefits are realized. For example, the longitudinally inward folded portion of the front waist region 22 ensures that none of the side seam 68 and the fastener 60 is exposed to the wearer's skin, reducing undesirable skin irritation. Moreover, in such a configuration, the fastener 60 is subjected to shear forces in use, such that the diaper 20 is more securely fastened upon the wearer. Finally, the fasteners 60, permanently bonded or attached to the back waist region 24 of the diaper 20, refastenably engage the diaper 20 in the front waist region 22 increasing the ease with which the wearer or the caregiver can adjust the fit of the diaper 20.

The waist size adjustment means provides improved fit and securement of the diaper 20 about the waist of the wearer after the diaper 20 has been placed on the wearer in the prefastened state. The waist size adjustment means is configured to reduce the waist perimeter dimension of the waist opening 33 to further conform the waist opening 33 to the waist of the wearer after initial donning of the diaper 20. As shown in FIG. 6, the second engageable portion 63 of the continuous fastener 60 extends from the prefastened, refastenable side seam 68 and is desirably configured to refastenably engage the attachment panel 66 and/or the exterior surface 36 of the front waist region 22. Alternatively, the second engageable portion 63 of the continuous fastener 60 may be configured to refastenably engage the exterior surface 36 of the back waist region 24.

Desirably, but not necessarily, the laterally opposing fasteners 60 are prefastened to the attachment panel 66 at least about 2.0 inches apart during the manufacturing process. With the laterally opposing fasteners 60 positioned at least about 2.0 inches apart, the initial donning of the diaper 20 is made easier without the need to unfastened the fasteners 60. In such a configuration, the prefastened diaper 20 can be pulled on and/or off over the legs and hips of the wearer, similar to conventional training pants. After the initial donning of the diaper 20, at least one of the refastenable fastener 60 provides the waist size adjustment means for adjusting the waist perimeter dimension, which corresponds to a peripheral or circumferential measurement of the waist opening 33, as shown in FIGS. 1 and 4. With the diaper 20 positioned about the waist of the wearer, the waist perimeter dimension may be adjusted by unfastening at least one of the fasteners 60 from the attachment panel 66 and refastening the at least one fastener 60 at a lateral position along the attachment panel 66 to either reduce or increase the waist perimeter dimension corresponding to the waist opening 33.

The prefastened, refastenable side seams 68 can also be easily disengaged to inspect the diaper 20 for possible soiling or to first apply the diaper 20 to the wearer if desired. If the prefastened diaper 20 becomes soiled during use, the prefastened, refastenable side seams 68 can be disengaged to easily remove the diaper 20 in a similar manner as conventional diaper articles.

In accordance with one embodiment of this invention, as shown in FIGS. 1 and 4, the prefastened, refastenable side seams 68 on the diaper 20 may further include passive bonds 70 (which, themselves, are not refastenable) for maintaining the diaper 20 in the prefastened condition, particularly when it is being pulled over the hips of the wearer. Desirably, the passive bonds 70 are easily broken or destroyed to release or disconnect the front waist region 22 from the back waist region 24. Absorbent articles including such passive bonds 70 and methods of making the passive bonds 70 are further described in U.S. Pat. No. 6,287,287, issued Sep. 11, 2001 to Elsberg on Jun. 19, 1998 and U.S. Pat. No. 6,036,805 issued Mar. 14, 2000 to McNichols, the disclosures of which are hereby incorporated by reference.

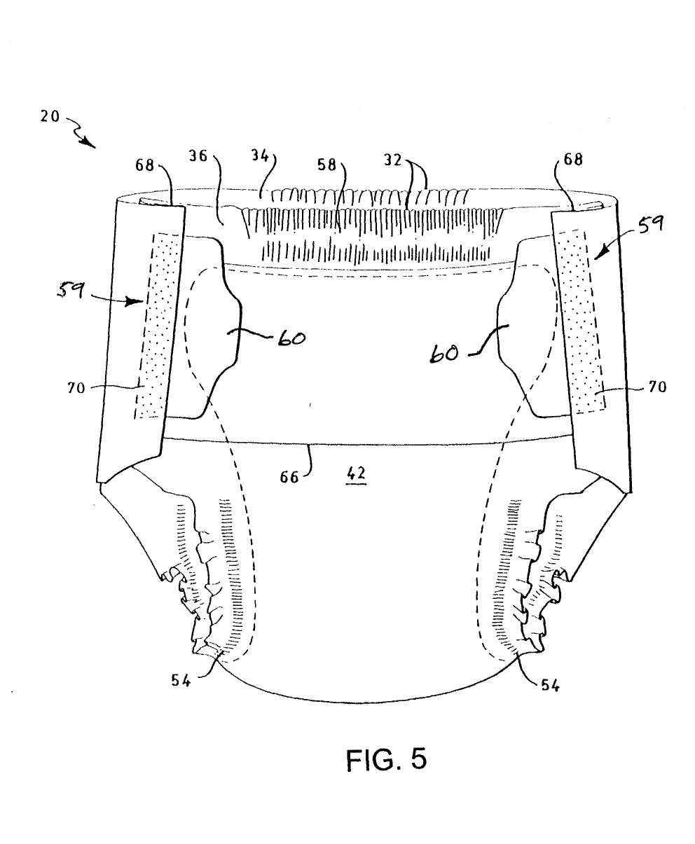

For example, as shown in FIGS. 4 and 5, the side seams 68 of the diaper 20 may include passive bonds 70 that releasably connect the side edges of the back waist region 24 to the front waist region 22 of the diaper 20. In such a configuration, the passive bonds 70 assist the first engageable portion 61 of the fasteners 60, that provide the refastenable side seams 68, in maintaining the diaper 20 in a prefastened configuration as the diaper 20 is pulled over the hips of the wearer. Moreover, the passive bonds 70 assist in securing the refastenable side seams 68 that prevent movement and shifting of the side edges 30 of the front waist region 22 and the back waist region 24 relative to each other for improved fit and performance. The passive bonds 70 also help maintain hip coverage and prevent rollover or folding of the side edges 30 and the waist edges 32 of the prefastened diaper 20 as the prefastened diaper 20 is pulled over the wearer's hips. Such prevention of rollovers and folding can reduce the level of contact between the fasteners 60 and the skin of the wearer, thus, resulting in reduced skin irritation and redness.

The passive bonds 70 may be located on the diaper 20 in any manner that provides the desired improved fastening while maintaining the desired elastic or extensible properties of the diaper components being bonded. For example, as shown in FIGS. 4 and 5, the passive bonds 70 may be located laterally inward of the side edges 30 of the front waist region 22 and/or the back waist region 24, adjacent to or included with the refastenable side seams 68. In such a configuration, the passive bonds 70 connect the front waist region 22 to the back waist region 24 of the diaper 20 in a facing relationship.

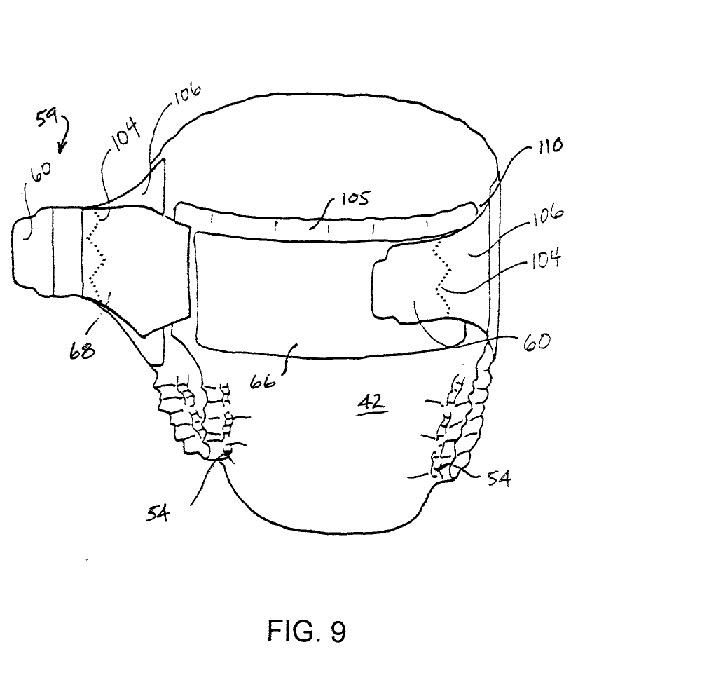

The passive bonds 70 can be provided by any type of bonding well known to those skilled in the art, such as thermal, adhesive and ultrasonic bonding, and may be discrete point bonds, dashed lines, continuous lines, discontinuous lines and the like or combinations thereof. Desirably, as shown in FIG. 9, the passive bonds 70 form a zig-zag bond pattern 104. In accordance with one embodiment of this invention, the zig-zag bond pattern 104 allows lateral side panels or ears 106 to elongate around a wearer's waist and to allow the side seam 68 to expand without the bonds opening or “zippering” from the stress of wear. Desirably, each side panel 106 is elastic or extensible in at least one direction, for example the cross-machine direction. The zig-zag bond pattern 104 has greater strength in the lateral direction (shear) than in the longitudinal direction (peel) in order to provide an easy opening joint if it is desired that the diaper 20 be opened along the side seams 68 for application or removal as a conventional diaper article. Desirably, the passive bonds 70 are ultrasonic point bonds, which can be destroyed upon the first opening of the diaper 20. Moreover, the passive bonds 70 may have any suitable shape such as circular, square, triangular and the like.

In accordance with one embodiment of this invention, the continuous fastener 60 may include a fastener substrate 84. Desirably, the fastener substrate 84 is made of an elastic or extensible material to provide improved fit and comfort to the wearer by allowing the continuous fastener 60 more flexibility and range in engaging the attachment panel 66 and/or the exterior surface 36 of the diaper 20. The fastener substrate 84 may be made of materials well known in the art. Suitable materials for the fastener substrate 84 include, but are not limited to, stretch-bonded-laminate (SBL) materials, neck-bonded-laminate (NBL) materials, elastomeric films, elastomeric foam materials, and the like, such as described above as being suitable for the fit panel 48.

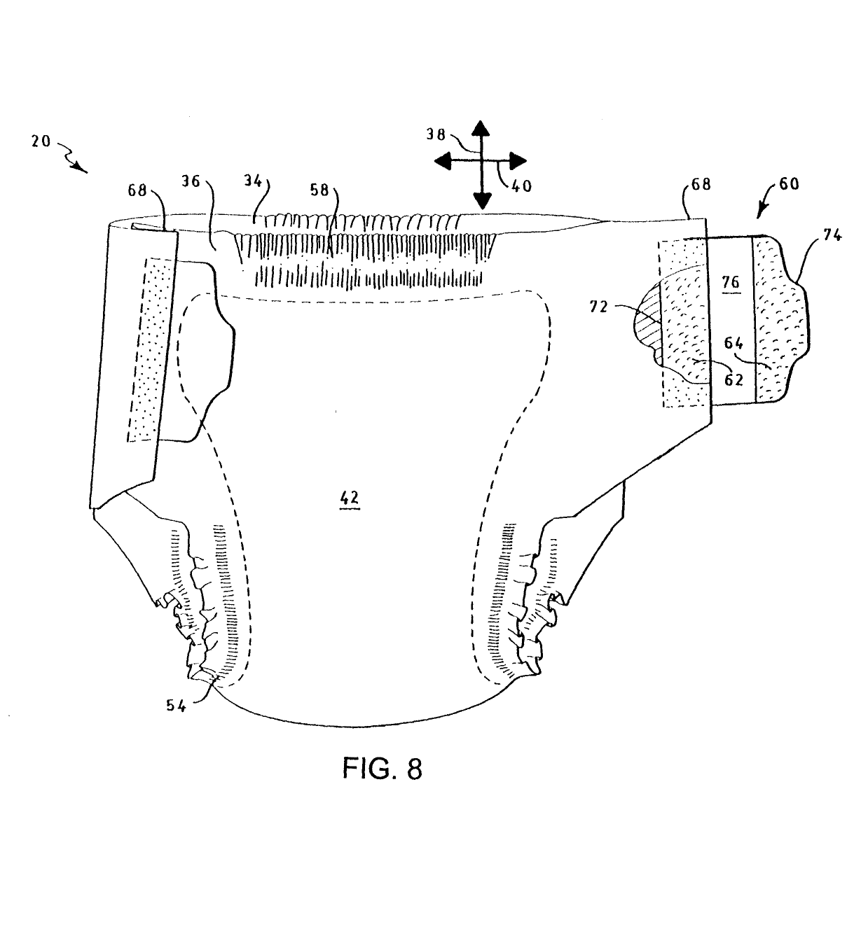

In accordance with one embodiment of this invention as shown in FIG. 8, each fastener 60 may include a primary fastener 62, and a waist size adjustment means, for example a secondary fastener 64. The primary fastener 62 may be permanently bonded or attached to the side edge 30 in one of the front waist region 22 and the back waist region 24 and refastenably attached to the side edge 30 in the opposite waist region 22 or 24 to provide the refastenable side seams 68.

The secondary fasteners 64 extend from the refastenable side seams 68 to form the waist size adjustment means. The secondary fasteners 64 are configured to encircle the hips of the wearer and engage the exterior surface 36 of the front waist region 22 of the diaper 20 or alternatively, the attachment panel 66, to reduce the waist perimeter dimension of the waist opening 33 and conform the waist opening 33 to the wearer's waist. It is apparent that the secondary fasteners 64 may alternatively be located on the front waist region 22 and may be configured to releasably engage the exterior surface 36 of the back waist region 24. Alternatively, the diaper 20 may include a single secondary fastener 64 extending from one of the refastenable side seams 68.

As shown in FIG. 8, the fasteners 60, when configured with multiple engaging portions, may include an intermediate portion 76 between the primary fastener 62 and the secondary 64 fastener. For instance, when the fasteners 60 include hook-type fastening elements in the multiple engaging portions, the intermediate portion may be devoid of hook-type fastening elements. Desirably, the intermediate portion 76 is made of an elastic or extensible material. Thus, the intermediate portion 76 provides improved fit and comfort to the wearer by allowing the fastener 60 more flexibility and range in engaging the attachment panel 66 and/or the exterior surface 36 of the diaper 20, either or both of which may include a loop fastening material. Suitable materials for the intermediate portion 76 include, but are not limited to, stretch-bonded-laminate (SBL) materials, neck-bonded-laminate (NBL) materials, elastomeric films, elastomeric foam materials, and the like, such as described above as being suitable for the fit panel 48.

In accordance with one embodiment of this invention, the intermediate portion 76 of the fastener 60 may include a hinge to which the primary fastener 62 and the secondary fastener 64 are attached. When the intermediate portion 76 is configured as a hinge, the fastener 60 desirably is thinner at the intermediate portion 76, and therefore more flexible for easier attachment of the secondary fastener 64 to the attachment panel 66 and/or the exterior surface 36 of the diaper 20.

The prefastened, refastenable side seams 68, which may include passive bonds 70, are configured to maintain the diaper 20 in a prefastened configuration as it is pulled on and off over the hips of the wearer during use. Thus, it is desirable that the prefastened refastenable side seams 68 provide adequate shear strength for maintaining the diaper 20 in the prefastened configuration. Such strengths are well known to those skilled in the art. Similarly, the waist size adjustment means, which may include the second engageable portion 63 of the continuous fastener 60 or the secondary fastener 64, should provide adequate shear strength and peel strength to maintain engagement with the exterior surface 36 of the diaper 20.

The side seams 68 are also configured to be readily broken such that the caregiver can easily peelingly disengage the front waist region 22 and the back waist region 24 to remove the diaper 20 from the wearer after it has been soiled, to inspect the diaper for soiling or to initially position the diaper 20 on the wearer if desired. Thus, it is desirable that the side seams 68 define a relatively low peel strength such that the caregiver can readily disengage the side seams 68 and break the passive bonds 70 to separate the front waist region 22 and the back waist region 24 to remove the diaper 20 from the waist of the wearer similar to conventional diapers that are not prefastened. Similarly, the waist size adjustment means that may include the second engageable portion 63 of the fastener 60 or the secondary fastener 64, are also configured to be readily broken such that the caregiver or the wearer can easily, peelingly disengage the waist size adjustment means from the exterior surface 36 of the diaper 20. This allows for the repositioning of the diaper 20 about the hips of the wearer, or for inspection of the diaper 20 without disengaging the side seams 68. Thus, it is desirable that the waist size adjustment means define a relatively low peel strength such that the caregiver can readily disengage the waist size adjustment means from the exterior surface 36 of the diaper 20. Suitable peel strength values are well known to those skilled in the art. One test for measuring peel strength of hook and loop fasteners is ASTM D1876-72. This test is described in U.S. Pat. No. 5,176,671, issued to Roessler et al., the disclosure of which is incorporated herein by reference.

### EXAMPLE

Referring to FIGS. 1-9, in accordance with one embodiment of this invention, a prefastened, refastenable diaper 20 includes an elastic waistband region 105, elastic back side panels or back ears 106, and an extensible outer cover 42. Additionally, the diaper 20 may include an elastic or extensible attachment panel 66 having loop-type fastener components. Table 1 below lists the required components for manufacturing the diaper 20 in accordance with one embodiment of this invention and the fit range provided or delivered by each component.

TABLE 1
Required Components

| Components Required | Fit Range Delivered (mm) |
|---|---|
| Elastic Back Side Panels or Ears | 102 |
| Elastic Waistband Region | 47 |
| Extensible Outer Cover | 136 |
| Extensible Attachment Panel having | 85 |
| loop-type fastener components | |
| Total Possible Fit Range | 370 |

In accordance with certain embodiments of this invention, it is desirable to have a diaper 20 having a minimum fit range of at least about 150 mm. A desired percent fit range of 33% is equal to the minimum fit range of 150 mm divided by the average relaxed waist performance measurement of 450 mm (for a Step 4 diaper).

The absorbent fastened, refastenable diapers according to this invention require additional components for manufacturing purposes and effective leakage reduction that do not reduce the fit range by tying up the elastic properties of the four components listed in Table 1. A minimum of 150 mm of fit range is required to make an absorbent, prefastened, refastenable diaper. Thus, additional components can be added in an effective manner as to not tie-up more than 220 mm of the potential 370 mm fit range (370 mm−150 mm=220 mm).

To maintain the minimum 150 mm fit range, a combination of the following components in Table 2 can be added in a manner that equals at least 220 mm, to provide a fit range that allows the absorbent article to be pulled over the hips and securely fastened around the waist of the wearer without readjustment to the fasteners.

The Percent Fit Range Delivered was calculated by dividing the Fit Range Delivered for each component by the relaxed waist performance measurement of 450 mm multiplied by 100. For example, for an extensible attachment panel the Percent Fit Range is equal to (85/450)×100, or 18.9%.

TABLE 2
Secondary Components

| | | Percent Fit |
|---|---|---|
| | Fit Range | Range |
| | Delivered | Delivered |
| Components | (mm) | (%) |
| Extensible or Elastic Attachment Panel | 85 | 18.9 |
| having loop-type fastener components | | |
| Containment flaps tacked down at least | 106 | 23.5 |
| 50 mm from the waistband region; | | |
| extensible containment flaps; or | | |
| containment flaps tacked down less than | | |
| about 35 mm in a lateral direction | | |
| Bellow in the Front Ear(s) | 44 | 9.7 |
| Elastic Back Ears attached to the back | 50 | 11.1 |
| waist region using attachment bonds | | |
| having a lateral or CD width of less than | | |
| 50 mm | | |
| Pleats in Tissue Wrapsheet | 20 | 4.4 |
| Back Waist Elastic Member applied | 136 | 30.2 |
| when stretched less than maximum | | |
| Front Waist Elastic Member applied | 85 | 18.9 |
| when stretched less than maximum | | |
| Non-extensible Spacer Layer positioned and | 68 | 15.1 |
| attached to the outer cover in the | | |
| waistband area without continuous glue | | |
| Extensible Spacer Layer | 68 | 15.1 |
| Fastener spacing less than 5 inches | 113 | 25.1 |

As displayed in Table 2, the suitable secondary components may include, but are not limited to: (a) an extensible or elastic fit panel 66 including loop-type fastener components; (b) containment flaps 56 which are tacked down at least about 50 mm from the waist band region 105; (c) at least one bellow 110, as shown in FIG. 9, which provides additional extensibility when the side panels or ears 106 are fastened to the attachment panel 66; (d) extensible back ears 106 which are bonded to the back waist region 24 of the diaper 20 using attachment bonds having a length less than about 50 mm; (e) pleats in the tissue wrapsheet 80; (f) a waist elastic member 58 attached to a back waist region 24 of the diaper 20 at less than maximum stretch; (g) a waist elastic member attached to a front waist region 22 of the diaper 20 at less than maximum stretch; (h) an extensible spacer layer positioned between the outer cover 42 and the absorbent core 28; (i) a non-extensible spacer layer that is not glued or otherwise connected to the outer cover 42; and () fasteners 60 having a spacing of less than about 5 inches between each other when fastened to the attachment panel 66.

For example, in accordance with one embodiment of this invention, in addition to the elastic waistband region 105 (47 mm), the extensible outer cover 42 (136 mm) and the elastic back ears 106 (102 mm), the diaper 20 may include as secondary components containment flaps 56 which are tacked down at least about 50 mm from the waist band region 105 (106 mm) and a waist elastic member 58 applied to the back waist region 24 of the diaper 20 at less than maximum stretch (136 mm). This combination of these secondary components will provide an additional fit range of at least about 242 mm.

Similarly, the diaper 20 may include as secondary components fasteners 60 positioned on the attachment panel 66 having a fastener spacing of less than about 5 inches (113 mm) and a waist elastic member 58 applied to the back waist region 24 of the diaper 20 at less than maximum stretch (136 mm). This combination of these secondary components will provide an additional fit range of at least about 249 mm.

While the invention has been described in detail with respect to specific aspects thereof, it will be appreciated that those skilled in the art, upon attaining an understanding of the foregoing, may readily conceive of alterations to, variations of and equivalents to these aspects. Accordingly, the scope of the present invention should be assessed as that of the appended claims and any equivalents thereto.

What is claimed is:

1. A prefastened, refastenable absorbent article, comprising:
an outer cover extensible in a lateral direction of the prefastened absorbent article and having a moisture barrier;
a bodyside liner at least partially bonded to the outer cover and extensible in the lateral direction; and
an absorbent core positioned between the outer cover and the body side liner;
the outer cover, bodyside liner and the absorbent core defining a chassis having a waist opening,
wherein the prefastened absorbent article has a percent fit range of at least about 33%.

2. The prefastened absorbent article of claim 1, wherein the percent fit range is about 80% to about 150%.

3. The prefastened absorbent article of claim 1, further comprising an elastic fit panel attached to the chassis.

4. The prefastened absorbent article of claim 1, wherein the chassis further comprises an elastic first side panel and a laterally opposing elastic second side panel.

5. The prefastened absorbent article of claim 4, further comprising a first fastener bonded to the first side panel and a laterally opposing second fastener bonded to the second side panel.

6. The prefastened absorbent article of claim 5, further comprising at least one attachment panel attached to a front waist region of the absorbent article, wherein each of the first fastener and the second fastener are releasably attached to the attachment panel.

7. The prefastened absorbent article of claim 6, wherein the attachment panel comprises a loop material, which is extensible in a lateral direction.

8. The prefastened absorbent article of claim 6, wherein in a prefastened condition, the first fastener and the second fastener are laterally separated by at least about 2.0 inches with respect to the attachment panel.

9. The prefastened absorbent article of claim 6, wherein the attachment panel is passively bonded to a back waist region of the absorbent article.

10. The prefastened absorbent article of claim 1, further comprising a spacer material at least partially bonded to the outer cover.

11. The prefastened absorbent article of claim 1, further comprising at least one extensible containment flap positioned along a first side edge of the prefastened absorbent article.

12. The prefastened absorbent article of claim 11, further comprising a second extensible containment flap positioned along a laterally opposing second side edge of the prefastened absorbent article.

13. The prefastened absorbent article of claim 1, further comprising a fastener feature having two laterally opposing hook-type fasteners prefastened to a loop component, wherein the two laterally opposing hook-type fasteners are spaced apart by at least about 2.0 inches in a lateral direction with respect to the loop component.

14. The prefastened absorbent article of claim 13, wherein the loop component is one of elastic and extensible in a lateral direction of the prefastened absorbent article.

15. The prefastened absorbent article of claim 1, further comprising a surge layer, the surge layer one of elastic and extensible in a lateral direction of the prefastened absorbent article.

16. The prefastened absorbent article of claim 1, comprising a diaper.

17. The prefastened absorbent article of claim 1, comprising training pants.

18. The prefastened absorbent article of claim 1, comprising absorbent underpants.

19. The prefastened absorbent article of claim 1, comprising an adult incontinence product.

20. The prefastened absorbent article of claim 1, comprising a feminine hygiene product.

21. The prefastened absorbent article of claim 1, comprising a health care garment.

22. A prefastened absorbent article, comprising:
a chassis having a front waist region, a back waist region and a crotch region intermediate the front waist region and the back waist region, the chassis defining a waist opening;
an outer cover having a moisture barrier;
a bodyside liner at least partially bonded to the outer cover; and
an absorbent core positioned between the outer cover and the bodyside liner.

23. The prefastened absorbent article of claim 22, wherein the chassis comprises an elastic material.

24. The prefastened absorbent article of claim 22, wherein the outer cover and the bodyside liner are one of elastic and extensible in the lateral direction.

25. The prefastened absorbent article of claim 22, wherein the outer cover and the bodyside liner are one of elastic and extensible in the longitudinal direction.

26. The prefastened absorbent article of claim 22, further comprising an extensible spacer layer positioned between the absorbent core and the outer cover.

27. The prefastened absorbent article of claim 26, wherein the spacer layer is partially bonded to the outer cover.

28. A pant-like prefastened, disposable absorbent article comprising:
an extensible outer cover;
an extensible bodyside liner connected to the outer cover in a superposed relation;
an absorbent core positioned between the outer cover and the bodyside liner;
an extensible spacer layer positioned between the backsheet and the absorbent core;
at least one extensible containment flap positioned adjacent a side edge of the absorbent core; and
at least one waist size adjustment means having a fastener and a corresponding loop component, the loop component extensible in a cross machine direction to permit one of a front waist region and a rear waist region to extend laterally beyond a width of a chassis,
wherein the pant-like disposable absorbent article has a percent fit range of at least about 33%.

29. A pant-like prefastened, disposable absorbent article comprising:
an extensible outer cover;
an elastic waistband region;
a first elastic back ear bonded to a back waist region of the pant-like disposable absorbent article;
an opposing second elastic back ear bonded to the back waist region; and
at least one secondary component, the at least one secondary component providing an additional fit range of at least about 220 mm.

30. The pant-like prefastened, disposable absorbent article of claim 29 wherein the at least one secondary component is selected from the group consisting of an extensible or elastic fit panel including loop-type fastener components, at least one bellow, tissue wrapsheet pleats, an extensible spacer layer, and fasteners having a fastener spacing of less than about 5 inches.

31. The pant-like prefastened, disposable absorbent article of claim 29 wherein the back ears are bonded to a back waist region of the absorbent article using attachment bonds having a length less than about 50 mm.

32. The pant-like prefastened, disposable absorbent article of claim 29 wherein the at least one secondary component comprises a waist elastic member attached to a back waist region of the absorbent article at less than maximum stretch.

33. The pant-like prefastened, disposable absorbent article of claim 29 wherein the at least one secondary component comprises a waist elastic member attached to a front waist region of the absorbent article at less than maximum stretch.

34. The pant-like prefastened, disposable absorbent article of claim 29 wherein the at least one secondary component comprises an extensible spacer layer positioned between an outer cover and an absorbent core.

35. The pant-like prefastened, disposable absorbent article of claim 34 wherein the spacer layer is not connected to the outer cover.

36. The pant-like prefastened, disposable absorbent article of Claim 29 wherein the at least one secondary component comprises at least one containment flap, the at least one containment flap tacked down to a chassis at least about 50 mm longitudinally from the elastic waistband region.

37. The pant-like prefastened, disposable absorbent article of Claim 29 wherein the at least one secondary component comprises a pleated tissue wrapsheet, the pleated tissue wrapsheet connected to an extensible bodyside liner of the absorbent article.
